(19) United States
(12) Patent Application Publication
(10) Pub. No.: US 2012/0318263 A1
(43) Pub. Date: Dec. 20, 2012

(54) ANESTHESIA VAPORIZER SYSTEM AND METHOD

(75) Inventors: Michael Eric Jones, Madison, WI (US); Robert Q. Tham, Madison, WI (US); Dave T. Clark, Madison, WI (US)

(73) Assignee: GENERAL ELECTRIC COMPANY, Schenectady, NY (US)

(21) Appl. No.: 13/161,900

(22) Filed: Jun. 16, 2011

(51) Int. Cl. A61M 16/01

(52) U.S. Cl. 128/203.12

(57) ABSTRACT

An anesthetic vaporizer system is disclosed herein. The anesthetic vaporizer system includes a pump, and a controller operatively connected to the pump. The controller is configured to generally simultaneously control both a frequency and a stroke of the pump in order to regulate the delivery of an anesthetic agent in the liquid phase. The anesthetic vaporizer system also includes a vaporization chamber in fluid communication with the pump. The vaporization chamber is adapted to vaporize the anesthetic agent delivered by the pump.

### FIELD OF THE INVENTION

This disclosure relates generally to an anesthesia vaporizer system and method. More specifically, this disclosure relates to a vaporizer system adapted to regulate the delivery of an anesthetic agent in the liquid phase.

### BACKGROUND OF THE INVENTION

Anesthesia may be administered to a patient in the form of a gas to produce an effect such as pain management, unconsciousness, preventing memory formation, and/or paralysis. A predetermined dosage of the administered anesthetic agent may be inhaled into the patient's lungs to produce one or more of these effects.

Anesthesia delivery systems may include an anesthesia machine pneumatically coupled with a vaporizer system. Conventional vaporizer systems regulate anesthetic agent dosage in the gas phase. More precisely, some conventional vaporizer systems raise the temperature of the anesthetic agent to its vaporization point and thereafter regulate the concentration of delivered anesthetic agent gas such that the output concentration is maintained at a preselected target concentration. Other anesthetic agents use a split flow vaporizer principle to control the concentration of the anesthetic agent.

One problem with some vaporizer systems is that environmental conditions such as pressure and temperature can impair the accuracy of anesthetic agent measurements in the gas phase. Another problem with some vaporizer systems is that they require significant power to heat and maintain the anesthetic agent at its vaporization temperature. Yet another problem with some vaporizer systems is that they have a startup lag associated with the time required to heat the anesthetic agent to its vaporization temperature. Finally, some conventional vaporizer systems are inefficient to manufacture and implement because they are dedicated for use exclusively with a single anesthetic agent.

### BRIEF DESCRIPTION OF THE INVENTION

The above-mentioned shortcomings, disadvantages and problems are addressed herein which will be understood by reading and understanding the following specification.

In an embodiment, an anesthetic vaporizer system includes a pump, and a controller operatively connected to the pump. The controller is configured to generally simultaneously control both a frequency and a stroke of the pump in order to regulate the delivery of an anesthetic agent in the liquid phase. The anesthetic vaporizer system also includes a vaporization chamber in fluid communication with the pump. The vaporization chamber is adapted to vaporize the anesthetic agent delivered by the pump.

In another embodiment, a method includes transferring a liquid anesthetic agent to a pump, and implementing a pump to deliver the liquid anesthetic agent to a vaporization chamber. The method also includes implementing a controller to generally simultaneously control both the frequency and the stroke of the pump in order to regulate the delivery of liquid anesthetic agent.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating an anesthesia system in accordance with an embodiment; and

FIG. 2 is a schematic diagram illustrating a vaporizer system in accordance with an embodiment

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

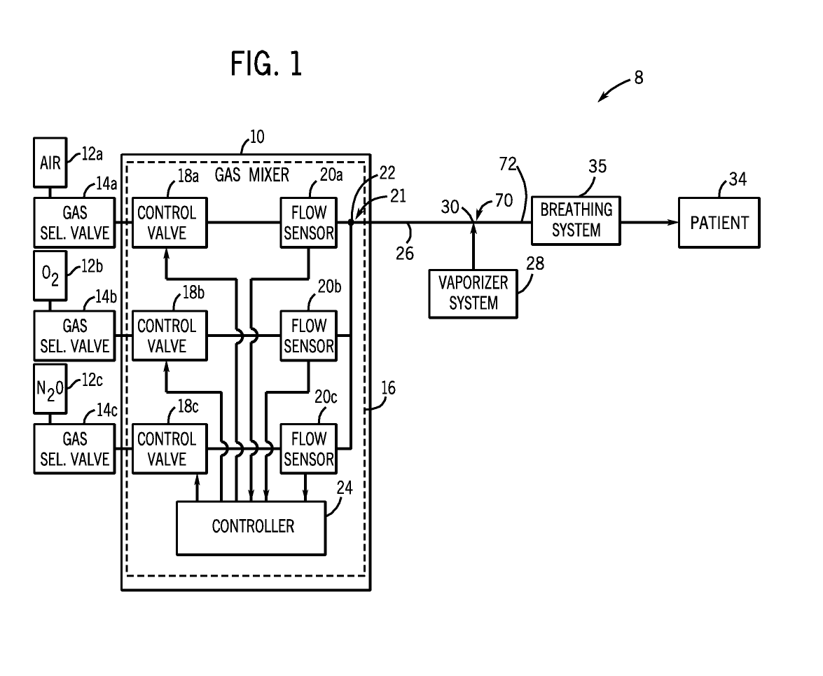

Referring to FIG. 1, an anesthesia system 8 is schematically depicted in accordance with one embodiment. The anesthesia system 8 includes an anesthesia machine 10, a plurality of gas storage devices 12a, 12b and 12c, and an anesthetic vaporizer system 28. The anesthesia machine 10 is shown for illustrative purposes and it should be appreciated that other types of anesthesia machines may alternately be implemented. In a typical hospital environment, the gas storage devices 12a, 12b and 12c are centrally located storage tanks configured to supply medical gas to multiple anesthesia machines and multiple hospital rooms. The storage tanks are generally pressurized to facilitate the transfer of the medical gas to the anesthesia machine 10.

The gas storage devices 12a, 12b and 12c will hereinafter be described as including an air tank 12a, an oxygen (O₂) tank 12b, and a nitrous oxide (N₂O) tank 12c, respectively, however it should be appreciated that other storage devices and other types of gas may alternatively be implemented. The gas storage tanks 12a, 12b and 12c are each connected to one of the gas selector valves 14a, 14b, and 14c, respectively. The gas selector valves 14a, 14b and 14c may be implemented to shut off the flow of medical gas from the storage tanks 12a, 12b and 12c when the anesthesia machine 10 is not operational. When one of the gas selector valves 14a, 14b and 14c is opened, gas from a respective storage tank 12a, 12b and 12c is transferred under pressure to the anesthesia machine 10.

The anesthesia machine 10 includes a gas mixer 16 adapted to receive medical gas from the storage tanks 12a, 12b and 12c. The gas mixer 16 includes a plurality of control valves 18a, 18b and 18c that are respectively connected to one of the gas selector valves 14a, 14b and 14c. The gas mixer 16 also includes a plurality of flow sensors 20a, 20b and 20c that are each disposed downstream from a respective control valve 18a, 18b, and 18c. After passing through one of the control valves 18a, 18b and 18c, and passing by one of the flow sensors 20a, 20b and 20c, the individual gasses (i.e., air, O₂ and N₂O) are combined to form a carrier gas 21 at the carrier gas outlet 22.

The control valves 18a, 18b and 18c and the flow sensors 20a, 20b and 20c are each connected to a controller 24. The controller 24 is configured to operate the control valves 18a, 18b and 18c in a response to gas flow rate feedback from the flow sensors 20a, 20b and 20c. Accordingly, the controller 24 can be implemented to maintain a selectable flow rate for each gas (i.e., air, O₂ and N₂O) such that the carrier gas 21 at the carrier gas outlet 22 comprises a selectable ratio of air, O₂ and N₂O.

The carrier gas 21 flows to a pneumatic circuit 26. The vaporizer system 28 introduces vaporized anesthetic agent 70 into the pneumatic circuit 26 at an inlet 30. Gas within the pneumatic circuit 26 disposed upstream relative to the inlet 30 comprises exclusively carrier gas 21. Gas within the pneumatic circuit 26 disposed downstream relative to the inlet 30 comprises a mixture of carrier gas 21 and vaporized anesthetic agent 70 and is therefore referred to as mixed gas 72. The mixed gas 72 is delivered to a patient 34 through a breathing system 35. Although the vaporizer system 28 is schematically depicted as being a separate component of the anesthesia system 8, it should be appreciated that it may alternatively be incorporated into the design of the anesthesia machine 10.

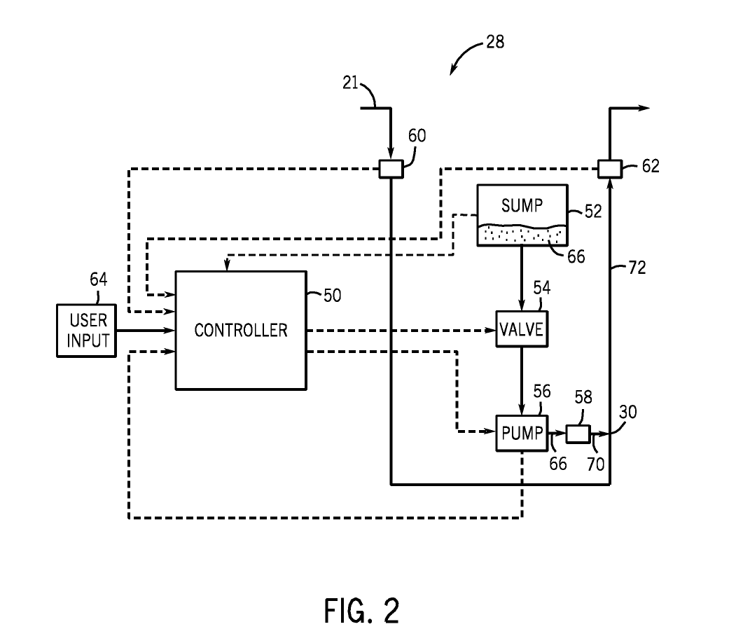

Referring to FIG. 2, the vaporizer system 28 is schematically depicted in accordance with an embodiment. The vaporizer system 28 may comprise a controller 50, a sump 52, a valve 54, a pump 56, a vaporization chamber 58, a carrier gas sensor 60 and/or a mixed gas sensor 62.

The controller 50 may be operatively connected to and adapted to receive input signals from a user input 64, the sump 52, the pump 56, the carrier gas sensor 60 and the mixed gas sensor 62. The user input 64 may comprise any device adapted to facilitate the transfer of information such as, for example, a keyboard, mouse, touchscreen, dial, trackball, voice recognition device, etc. The controller 50 may be operatively connected to and adapted to transmit output signals to the valve 54 and the pump 56. According to one embodiment, the controller 50 may comprise a computer.

The sump 52 is adapted to retain a liquid anesthetic agent 66. According to one embodiment the liquid anesthetic agent 66 may comprise desflurane, however other anesthetic agents may alternatively be implemented. Those skilled in the art will appreciate that desflurane has a boiling point of 23.5 degrees Celsius at 1 atmosphere pressure such that it can vaporize at or near room temperature. It may therefore be desirable to implement a thermal regulation system and/or pressure regulation system to maintain the anesthetic agent 66 in its liquid phase. Operating conditions such as temperature, pressure, and/or liquid level of the liquid in the sump 52 can be sensed and communicated to the controller 50.

The valve 54 is selectively operable in an open position or mode in which liquid anesthetic agent 66 is transferrable from the sump 52 to the pump 56. The valve 54 is also selectively operable in a closed position or mode in which liquid anesthetic agent 66 is precluded from being transferred from the sump 52 to the pump 56. The controller 50 may be implemented to select the operation mode of the valve 54. As an example, the controller 50 may regulate operation of the valve 54 such that liquid anesthetic agent 66 is transmitted to the pump 56 for delivery to the patient 34 (shown in FIG. 1) in response to a command from the user input 64.

The pump 56 may comprise a variety of different types of pump such as, for example, a solenoid driven microfluidic pump or a piezoelectric microfluidic pump. The pump 56 may have a variable frequency that is adjustable within a predefined range to regulate volumetric delivery of the liquid anesthetic agent 66. Those skilled in the art will appreciate that the frequency of the pump 56 is a measure of the pump's operational speed which may be measured in oscillatory cycles per second. The pump 56 may also have a variable stroke length that is adjustable within a predefined range to regulate volumetric delivery of the liquid anesthetic agent 66. Those skilled in the art will appreciate that the stroke length of the pump 56 is a measure of the mechanical deflection of the pump's operating mechanism (e.g., an elastic diaphragm or piston). In addition, unidirectional valves (not shown) can be incorporated to pump 56 along the liquid flow passages to direct liquid anesthetic agent 66 towards the patient 34 only, and to prevent retrograde flow toward the sump 52. Alternate schemes, such as pressurizing the liquid upstream of the pump 56 above the differential pressure generated by the pump 56, to prevent retrograde flow of liquid anesthetic agent 66 is also anticipated.

The vaporization chamber 58 is adapted to convert liquid anesthetic agent 66 from the pump 56 into vaporized anesthetic agent 70. The vaporization chamber 58 may comprise a heat source (not shown) adapted to raise the temperature of the liquid anesthetic agent 66 and thereby facilitate its conversion to vaporized anesthetic agent 70. The heat source may, for example, comprise a heated resistive wire, a cartridge heater, a peltier device, a sintered heater, or a passive heating system such as a system comprising heat pipes. Vaporized anesthetic agent 70 from the vaporization chamber 58 is delivered to the inlet 30 and is then mixed with the carrier gas 21 to form mixed gas 72. In an alternative embodiment, the carrier gas 21 may be fed directly to the vaporization chamber 58 to facilitate the vaporization of the liquid anesthetic agent, and improve the efficiency to mix and transport the vaporized anesthetic agent 70 to form the mix gas 72.

The carrier gas sensor 60 and the mixed gas sensor 62 may comprise a known device adapted to measure characteristic features of a fluid. For purposes of this disclosure the term fluid should be defined to include any amorphous substance that continually deforms under an applied shear stress and may therefore include both liquids and gases.

According to one embodiment, the carrier gas sensor 60 is adapted to measure fluid flow rate, and the mixed gas sensor 62 is configured to measure vaporized anesthetic agent 70 concentrations. In another embodiment, both the carrier gas sensor 60 and the mixed gas sensor 62 are configured to measure fluid flow rate, and the comparison of the fluid flow rates provide the measured concentration output of the anesthetic vaporizer 28, and the amount of liquid anesthetic agent 66 to be delivered by the pump 56 to achieve the concentration output of the anesthetic vaporizer 28. In yet another embodiment, the vaporized anesthetic agent 70 concentration can be measured directly by sensor 62 configured to detect the concentration of the vaporized anesthetic 70 concentrations. Known technology that measures vapor concentrations of liquid anesthetic include but are not limited to infrared, ultrasound, mass spectroscopy, and laser refractometry technologies. Vapor anesthetic concentration can also be measured based on feedback from sensors 60 and 62 respectively configured to assess any physical property (e.g., gas density) of the carrier gas 21 and the mixed gas 72. A feedback control system incorporating the measured vaporized anesthetic agent 70 concentrations and the user input 64 concentrations can be configured to adjust the operation of pump 56 to achieve the user set vaporizer output concentration.

Having described exemplary components of the vaporizer system 28, the operation of the vaporizer system 28 will now be described in accordance with an embodiment.

The controller 50 may be adapted to receive a target dosage or concentration of anesthetic agent from the user input 64. Upon receipt of the user requested target concentration, the controller 50 may open the valve 54 to facilitate the transfer of liquid anesthetic agent 66 from the sump 52 to the pump 56. The controller 50 may then regulate the pump 56 to deliver the liquid anesthetic agent 66 flow rate in a manner adapted to achieve the user requested target dosage or concentration of anesthetic agent. The liquid anesthetic agent 66 may then be converted to vaporized anesthetic agent 70 by the vaporization chamber 58. The vaporized anesthetic agent 70 is delivered to the inlet 30 and is then mixed with the carrier gas 21 to form mixed gas 72. The mixed gas 72 comprising the user selected target concentration of vaporized anesthetic agent 70 may then be delivered to the patient 34 (shown in FIG. 1).

According to another embodiment, the controller 50 may be adapted to operate the valve 54 and the pump 56 based on feedback from the carrier gas sensor 60 and/or the mixed gas sensor 62. As an example, the controller 50 may operate valve 54 and pump 56 to adjust the flow delivery of the liquid anesthetic agent 66 based on the carrier gas flow rate (as measured by the carrier gas sensor 60) and/or the mixed gas flow rate (as measured by the mixed gas sensor 62). As another example, the controller 50 may operate valve 54 and pump 56 to adjust the flow delivery of the liquid anesthetic agent 66 based on the measured concentration of vaporized anesthetic agent 70 in the mixed gas 72 (as measured by the mixed gas sensor 62).

The pump 56 should be controllable in a manner adapted to maintain a generally constant flow rate of liquid anesthetic agent 66 such that the delivery of vaporized anesthetic agent 70 to the patient is uniform over the delivery interval. More precisely, it has been established that the flow rate should be maintainable at a selectable constant value within a +/−0.01 μl/sec margin of error. The pump 56 should also be controllable in a manner adapted to provide a variable liquid anesthetic agent flow rate within a range of 0.02 μl/sec to 250 μl/sec such that the concentration of vaporized anesthetic agent 70 to the patient is maintained within a range of approximately 1% to 18% for desflurane, 0.2% to 5% for halothane and isoflurane, 0.2% to 8% for sevoflurane, and at a carrier gas flow rate that range between 0.2 l pm to 15 l pm. Clinical feedback has established that for a universal vaporizer design to be adapted to deliver any of the volatile anesthetic agents that can be customized for different anesthetic agents, an agent concentration dosage within the range of approximately 0.2% to 18% is appropriate for virtually all patient treatment.

The pump 56 delivers liquid flow rate as a product of the stroke volume and stroke frequency. It has been observed that liquid anesthetic agent delivery consistency and precision are best be maintained by generally simultaneously controlling both the frequency and the stroke of the pump 56. If the pump 56 is controlled only by stroke volume or frequency, the required dynamic range of the controlled parameter is 1:12,500 with an incremental step of 1 in 25,000 or better. Such a large range in stroke volume can compromise the accuracy or resolution of the overall delivery. If the vaporizer is controlled by frequency alone, to accommodate a smooth low ripple vaporizer 28 output delivery the fixed stroke volume must be small (tens of nanoliters or less) and delivered at multiple stroke frequency per second. At this fixed stroke volume, a high operating frequency (at least 12,500 times faster than the lowest stroke frequency) is required to deliver at the highest liquid flow rate. Such high oscillating frequency can impose significant repetitive wear on the pump and compromise reliability. With simultaneous control of both stroke volume and frequency, the enormous dynamic range can be apportioned and combined as a product of these two parameters. For example, a much less demanding dynamic range of 100 in stroke volume and 125 in frequency will yield the desired 1:12,500 dynamic range of the vaporizer design. Algorithms to adapt these parameters to deliver the anesthetic flow rate and to optimize performance such as delivery resolution, accuracy, rapid response to large step changes in vapor flow delivery, and minimize hysteresis, pump wear, power usage may reside with the controller 50.

For at least the previously described reasons the controller 50 may be configured to generally simultaneously control both the frequency and the stroke of the pump 56 to regulate delivery of liquid anesthetic agent 66. The resultant anesthetic agent delivery is sufficiently consistent and precise for its intended use. The resultant flow rate range is wide enough to deliver any currently available anesthetic agent compound. The ability to deliver any currently available anesthetic agent compound renders the vaporizer system 28 more efficient to manufacture and implement as compared to conventional vaporizer systems dedicated for use exclusively with a single anesthetic agent.

It should be appreciated that, by controlling the pump 56 in the manner previously described, the vaporizer system 28 regulates the dosage of the vaporized anesthetic agent 72 while in its liquid anesthetic agent 66 phase. Regulating the dosage of the anesthetic agent 66 in its liquid phase minimizes the impact of environmental conditions such as pressure and temperature, and thereby improves the precision with which a given dosage can be administered. Regulating the dosage of the anesthetic agent 66 in its liquid phase also reduces power requirements that would otherwise be necessary to maintain an anesthetic agent at its vaporization temperature such that the vaporizer system 28 is more energy efficient. Regulating the dosage of the anesthetic agent 66 in its liquid phase also reduces or eliminates the startup lag associated with conventional vaporizers that heat an anesthetic agent to its vaporization temperature before it is regulated and delivered.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. An anesthetic vaporizer system comprising:
a pump;
a controller operatively connected to the pump, said controller configured to generally simultaneously control both a frequency and a stroke of the pump in order to regulate the delivery of an anesthetic agent in the liquid phase; and
a vaporization chamber in fluid communication with the pump, said vaporization chamber adapted to vaporize the anesthetic agent delivered by the pump.

2. The anesthetic vaporizer system of claim 1, wherein the controller is configured to generally simultaneously control both a frequency and a stroke of the pump such that the flow rate of the anesthetic agent is maintainable at a constant value within a +/−0.01 μl/sec margin of error.

3. The anesthetic vaporizer system of claim 1, wherein the controller is configured to generally simultaneously control both a frequency and a stroke of the pump such that the flow rate of the anesthetic agent is variable within a range of 0.02 μl/sec to 250 μl/sec.

4. The anesthetic vaporizer system of claim 1, wherein the controller is configured to generally simultaneously control both a frequency and a stroke of the pump based on a measured carrier gas flow rate.

5. The anesthetic vaporizer system of claim 1, wherein the controller is configured to generally simultaneously control both a frequency and a stroke of the pump based on a measured mixed gas flow rate.

6. The anesthetic vaporizer system of claim 1, wherein the controller is configured to generally simultaneously control both a frequency and a stroke of the pump based on a measured anesthetic agent concentration.

7. The anesthetic vaporizer system of claim 1, wherein the controller is configured to generally simultaneously control both a frequency and a stroke of the pump based a user input command.

8. The anesthetic vaporizer system of claim 1, wherein the pump comprises a solenoid driven microfluidic pump or a piezoelectric microfluidic pump.

9. A method comprising:
transferring a liquid anesthetic agent to a pump;
implementing a pump to deliver the liquid anesthetic agent to a vaporization chamber; and
implementing a controller to generally simultaneously control both the frequency and the stroke of the pump in order to regulate the delivery of liquid anesthetic agent.

10. The method of claim 9, further comprising implementing a controller to generally simultaneously control both a frequency and a stroke of the pump such that the flow rate of the anesthetic agent is maintainable at a constant value within a +/−0.01 μl/sec margin of error.

11. The method of claim 9, further comprising implementing a controller to generally simultaneously control both a frequency and a stroke of the pump such that the flow rate of the anesthetic agent is variable within a range of 0.02 μl/sec to 250 μl/sec.

12. The method of claim 9, further comprising implementing a controller to generally simultaneously control both a frequency and a stroke of the pump based on a measured carrier gas flow rate.

13. The method of claim 9, further comprising implementing a controller to generally simultaneously control both a frequency and a stroke of the pump based on a measured mixed gas flow rate.

14. The method of claim 9, further comprising implementing a controller to generally simultaneously control both a frequency and a stroke of the pump based on a measured anesthetic agent concentration.

15. The method of claim 9, further comprising implementing the vaporization chamber to convert the liquid anesthetic agent into a vaporized anesthetic agent.

16. The method of claim 15, further comprising delivering the vaporized anesthetic agent to a patient.
